(19) United States
(12) Patent Application Publication
(10) Pub. No.: US 2019/0282208 A1
(43) Pub. Date: Sep. 19, 2019

(54) METHODS AND APPARATUSES FOR GENERATING AND DISPLAYING ULTRASOUND IMAGES USING AN EXPLAINING MODEL

(71) Applicants: Nathan Silberman, Brooklyn, NY (US); Pouya Samangouei, New York, NY (US)

(72) Inventors: Nathan Silberman, Brooklyn, NY (US); Pouya Samangouei, New York, NY (US)

(73) Assignee: Butterfly Network, Inc., Guilford, CT (US)

(21) Appl. No.: 16/352,424

(22) Filed: Mar. 13, 2019

Related U.S. Application Data
(60) Provisional application No. 62/692,370, filed on Jun. 29, 2018.
(60) Provisional application No. 62/643,120, filed on Mar. 14, 2018.

(51) Int. Cl. A61B 8/00; G06T 7/10; G09B 23/28

(52) U.S. Cl. CPC A61B 8/461; G06T 2207/30004; G09B 23/28; G06T 7/10

(57) ABSTRACT

Aspects of the technology described herein relate to collection and display of ultrasound images using an explaining model. A first ultrasound image may be determined to be in a first class and a second ultrasound image that is in a second class may be generated based on the first ultrasound image. The second ultrasound image may be generated by an explaining model. A classification model may classify the first and second ultrasound images in the first and second classes, respectively. Generating the second ultrasound image may include changing one or more portions of the first ultrasound image. The explaining model may also generate a transformed version of the first ultrasound image and a mask image, and the second ultrasound image may be a composite image of the first ultrasound image and the transformed version of the first ultrasound image. The mask image may determine how to generate the composite image.

### CROSS-REFERENCE TO RELATED APPLICATIONS

The present application claims the benefit under 35 USC § 119(e) of U.S. Application Ser. No. 62/692,370, filed Jun. 29, 2018, under Attorney Docket No. B1348.70076US01 and entitled “METHODS AND APPARATUSES FOR GENERATING AND DISPLAYING ULTRASOUND IMAGES USING AN EXPLAINING MODEL,” which is hereby incorporated herein by reference in its entirety.

The present application claims the benefit under 35 USC § 119(e) of U.S. Application Ser. No. 62/643,120, filed Mar. 14, 2018, under Attorney Docket No. B1348.70076US00 and entitled “MODEL EXPLANATION VIA DECISION BOUNDARY CROSSING TRANSFORMATIONS,” which is hereby incorporated herein by reference in its entirety.

### FIELD

Generally, the aspects of the technology described herein relate to collection and display of ultrasound images. Some aspects relate to collection and display of ultrasound images using an explaining model.

### BACKGROUND

Ultrasound devices may be used to perform diagnostic imaging and/or treatment, using sound waves with frequencies that are higher with respect to those audible to humans. Ultrasound imaging may be used to see internal soft tissue body structures, for example to find a source of disease or to exclude any pathology. When pulses of ultrasound are transmitted into tissue (e.g., by using an ultrasound device), sound waves are reflected off the tissue, with different tissues reflecting varying degrees of sound. These reflected sound waves may then be recorded and displayed as an ultrasound image to the operator. The strength (amplitude) of the sound signal and the time it takes for the wave to travel through the body provide information used to produce the ultrasound image. Many different types of images can be formed using ultrasound devices, including real-time images. For example, images can be generated that show two-dimensional cross-sections of tissue, blood flow, motion of tissue over time, the location of blood, the presence of specific molecules, the stiffness of tissue, or the anatomy of a three-dimensional region.

### SUMMARY

According to one aspect, a method includes determining, with a processing device, that a classification model classifies a first ultrasound image as belonging to a first class; generating, based on the first ultrasound image, a second ultrasound image that the classification model would classify as belonging to a second class, wherein the second class is different from the first class; and displaying the second ultrasound image.

In some embodiments, generating the second ultrasound image includes changing one or more portions of the first ultrasound image. In some embodiments, generating the second ultrasound image includes inputting the first ultrasound image to an explaining model configured to accept the first ultrasound image as an input and output the second ultrasound image based on the first ultrasound image.

In some embodiments, determining that the classification model classifies the first ultrasound image as belonging to the first class includes inputting the first ultrasound image to the classification model. In some embodiments, the classification model is configured to classify the inputted ultrasound image according to a quality metric of the inputted ultrasound image. In some embodiments, the classification model is configured to classify the inputted ultrasound image according to an anatomical view shown in the inputted ultrasound image. In some embodiments, the explaining model is trained using ultrasound images classified by the classification model. In some embodiments, the classification model is configured to classify ultrasound images as belonging to either the first class or the second class.

In some embodiments, determining that the classification model classifies the first ultrasound image as belonging to the first class includes inputting the first ultrasound image to the classification model. In some embodiments, the classification model is configured to classify the inputted ultrasound image according to a quality of the inputted ultrasound image. In some embodiments, the classification model is configured to classify the inputted ultrasound image according to an anatomical view shown in the inputted ultrasound image. In some embodiments, the classification model is configured to classify ultrasound images as belonging to either the first class or the second class. In some embodiments, the first class includes a low-quality class and the second class includes a high-quality class. In some embodiments, classification of an ultrasound image as belonging to the low-quality class or the high-quality class is based on: a clinical use metric indicating a probability that a medical professional would use the respective image for clinical evaluation; and a segmentation metric indicating a confidence that a segmentation performed on the ultrasound image is correct. In some embodiments, the first class includes a first anatomical view and the second class includes a second anatomical view.

In some embodiments, generating the second ultrasound image includes generating a composite of the first ultrasound image and a transformed version of the first ultrasound image. In some embodiments, generating the composite of the first ultrasound image and the transformed version of the first ultrasound includes generating a weighted sum of the first ultrasound image and the transformed version of the first ultrasound image.

In some embodiments, the explaining model includes a generator, and the method further includes generating the transformed version of the first ultrasound image using the generator. In some embodiments, the explaining model further includes a first encoder, and the method further includes generating, using the first encoder, a hidden vector based on the first ultrasound image; and inputting the hidden vector to the generator. In some embodiments, the explaining model further includes a second encoder. In some embodiments, the method further includes generating a mask image indicating changes from the first ultrasound image to the second ultrasound image.

In some embodiments, generating the second ultrasound image includes generating a composite of the first ultrasound image and a transformed version of the first ultrasound image; generating the composite of the first ultrasound image and the transformed version of the first ultrasound image includes generating a weighted sum of the first ultrasound image and the transformed version of the first ultrasound image; and the mask image determines the weighted sum. In some embodiments, the method further includes displaying the mask image. In some embodiments, the method further includes displaying the mask image and the second ultrasound image simultaneously. In some embodiments, the method further includes displaying the mask image, the second ultrasound image, and the first ultrasound image simultaneously. In some embodiments, the method further includes highlighting regions of the first ultrasound image and/or the second ultrasound image based on the mask image. In some embodiments, the explaining model includes a generator, and the method further includes generating the transformed version of the first ultrasound image using the generator. In some embodiments, the explaining model further includes a first encoder, and the method further includes generating, using the first encoder, a hidden vector based on the first ultrasound image; and inputting the hidden vector to the generator. In some embodiments, the explaining model further includes a second encoder.

In some embodiments, the method further includes receiving the first ultrasound image from an ultrasound device. In some embodiments, receiving the first ultrasound image from the ultrasound device includes receiving the first ultrasound image in real-time. In some embodiments, the method further includes receiving the first ultrasound image from a memory. In some embodiments, generating the second ultrasound image is performed in response to receiving a user selection. In some embodiments, displaying the second ultrasound image is performed in response to receiving a first user selection. In some embodiments, displaying the first ultrasound image is performed in response to receiving a second user selection following the first user selection. In some embodiments, the classification model includes one or more convolutional neural networks. In some embodiments, the explaining model includes one or more convolutional neural networks.

Some aspects include at least one non-transitory computer-readable storage medium storing processor-executable instructions that, when executed by at least one processor, cause the at least one processor to perform the above aspect and embodiments. Some aspects include an ultrasound system having a processing device configured to perform the above aspect and embodiments.

### BRIEF DESCRIPTION OF THE DRAWINGS

Various aspects and embodiments will be described with reference to the following exemplary and non-limiting figures. It should be appreciated that the figures are not necessarily drawn to scale. Items appearing in multiple figures are indicated by the same or a similar reference number in all the figures in which they appear.

FIG. 1 illustrates an example process for guiding collection of ultrasound data, in accordance with certain embodiments described herein;

FIG. 2 illustrates an example graphical user interface (GUI) that may be displayed on a display screen of a processing device in an ultrasound system, in accordance with certain embodiments described herein. The GUI shows a collected ultrasound image;

FIG. 3 illustrates an example GUI that may be displayed on a display screen of a processing device in an ultrasound system, in accordance with certain embodiments described herein. The GUI shows a collected ultrasound image and an output of an explaining model;

FIG. 4 illustrates an example GUI that may be displayed on a display screen of a processing device in an ultrasound system, in accordance with certain embodiments described herein. The GUI shows a collected ultrasound image and an output of an explaining model in a different manner;

FIG. 5 illustrates an example GUI that may be displayed on a display screen of a processing device in an ultrasound system, in accordance with certain embodiments described herein. The GUI shows a collected ultrasound image and an output of an explaining model in a different manner;

FIG. 6 illustrates an example GUI that may be displayed on a display screen of a processing device in an ultrasound system, in accordance with certain embodiments described herein. The GUI shows a collected ultrasound image and an output of an explaining model in a different manner;

FIG. 7 illustrates an example GUI that may be displayed on a display screen of a processing device in an ultrasound system, in accordance with certain embodiments described herein. The GUI shows a collected ultrasound image;

FIG. 8 illustrates an example GUI that may be displayed on a display screen of a processing device in an ultrasound system, in accordance with certain embodiments described herein. The GUI shows a collected ultrasound image and an output of an explaining;

FIG. 9 illustrates an example architecture for an explaining model in accordance with certain embodiments described herein;

FIG. 10 illustrates example input images to and output images from an explaining model in accordance with certain embodiments described herein;

FIG. 11 illustrates more example input images to and output images from an explaining model in accordance with certain embodiments described herein;

FIG. 12 illustrates more example input images to and output images from an explaining in accordance with certain embodiments described herein;

FIG. 13 illustrates more example input images to and output images from an explaining in accordance with certain embodiments described herein;

### DETAILED DESCRIPTION

Ultrasound examinations often include the acquisition of ultrasound images that contain a view of a particular anatomical structure (e.g., an organ) of a subject. Acquisition of these ultrasound images typically requires considerable skill. For example, an ultrasound technician operating an ultrasound device may need to know where the anatomical structure to be imaged is located on the subject and further how to properly position the ultrasound device on the subject to capture a medically relevant ultrasound image of the anatomical structure. Holding the ultrasound device a few inches or centimeters too high or too low on the subject may make the difference between capturing a medically relevant ultrasound image and capturing a medically irrelevant ultrasound image. As a result, non-expert operators of an ultrasound device may have considerable trouble capturing medically relevant ultrasound images of a subject. Common mistakes by these non-expert operators include, for example: capturing ultrasound images of the incorrect anatomical structure and capturing foreshortened (or truncated) ultrasound images of the correct anatomical structure.

Conventional ultrasound systems are large, complex, and expensive systems that are typically only purchased by large medical facilities with significant financial resources. Recently, cheaper and less complex ultrasound devices have been introduced. Such imaging devices may include ultrasonic transducers monolithically integrated onto a single semiconductor die to form a monolithic ultrasound device. Aspects of such ultrasound-on-a chip devices are described in U.S. patent application Ser. No. 15/415,434 titled “UNIVERSAL ULTRASOUND DEVICE AND RELATED APPARATUS AND METHODS,” filed on Jan. 25, 2017 (and assigned to the assignee of the instant application), which is incorporated by reference herein in its entirety. The reduced cost and increased portability of these new ultrasound devices may make them significantly more accessible to the general public than conventional ultrasound devices.

The inventors have recognized and appreciated that although the reduced cost and increased portability of ultrasound devices makes them more accessible to the general populace, people who could make use of such devices have little to no training for how to use them. For example, a small clinic without a trained ultrasound technician on staff may purchase an ultrasound device to help diagnose patients. In this example, a nurse at the small clinic may be familiar with ultrasound technology and physiology, but may know neither which anatomical views of a patient need to be imaged in order to identify medically-relevant information about the patient nor how to obtain such anatomical views using the ultrasound device. In another example, an ultrasound device may be issued to a patient by a physician for at-home use to monitor the patient's heart. In all likelihood, the patient understands neither physiology nor how to image his or her own heart with the ultrasound device. Accordingly, the inventors have developed assistive ultrasound imaging technology for guiding an operator to capture medically relevant ultrasound data. For example, the assistive ultrasound imaging technology may include automatic classification of ultrasound images by a classification model. For example, the classification model may classify the quality of ultrasound images or anatomical views shown in the ultrasound images.

A conventional classification model may not make clear why it decides to classify data in a particular class. In particular, it may not be clear what high-level, semantic properties of the inputs (e.g., ultrasound images being classified) the classification model uses to discriminate between specific classes. As an example, if a classification model classifies an ultrasound image that a user collected with an ultrasound imaging device as low-quality, it may not be clear why the classification model produced this classification, and the user may thereby receive no insight how to better use the ultrasound imaging device to collect an ultrasound image that the classification model would classify as high-quality.

The inventors have recognized this shortcoming and addressed it by developing a post-hoc technique for explaining a classification model's decision boundary (where “post-hoc” means that the explanation does not require understanding the inner workings of the classification model). In particular, the inventors have developed a technique for visually explaining a classification model's decisions by producing, using an explaining model, images (e.g., ultrasound images) on either side of the classification model's decision boundary whose differences are perceptually clear. Such an approach may make it possible for a human to conceptualize how the classification model is making its decisions at the level of semantics or concepts, rather than vectors of pixels. The technique developed by the inventors for using an explaining model to visually explain a classification model's decisions improves ultrasound technology because it allows for the generation of higher-quality ultrasound images as compared to conventional techniques. Indeed, as described herein, the explaining model may enable a user to reposition the ultrasound probe, remove a part of an image having low quality, and/or discard low-quality images such that subsequent analyses are not degraded.

The technique includes the use of generative models that transform images from one domain to another. Given a pre-trained classification model, embodiments described herein introduce a second, post-hoc explaining network that takes an input image that falls on one side of the classification model's decision boundary and produces a changed version of the image that falls on the other side of the decision boundary.

Three properties contribute to making the explaining model helpful for post-hoc model interpretation:

1. Easily visualizable differences: The explaining model may change the input image in a manner that is clearly detectable by the human eye.

2. Localized differences: The explaining model may yield changes to the input image that are spatially localized. Such sparse changes may be more easily interpretable by a viewer.

3. Semantically consistent: The explaining model may be consistent with the behavior of the pre-trained classifier in that the pre-trained classifier predicts different labels for the input and changed images.

The explaining model may be useful for helping a user use an ultrasound imaging devices to collect ultrasound images of a particular class. For example, a classification model may classify the quality of ultrasound images as they are collected. If the classification model classifies an ultrasound image as low quality, a user may select an option to generate another ultrasound image, which may be similar to the collected ultrasound image but classified as in a high-quality class. As described above, the explaining model may generate the ultrasound image such that changes from the collected ultrasound image to the generated ultrasound image are visually perceptible. Accordingly, if a user views the collected ultrasound image and is unsure why the classification model classifies the ultrasound image as a low-quality image, the user may be able to see, from the generated ultrasound image, what changes to the collected ultrasound image may cause it to be classified in the high-quality class. The user may thereby gain insight into how to alter the current ultrasound image collection to collect a high-quality image. For example, the explaining model may indicate that certain anatomical structures, if removed from an ultrasound image, would transform the ultrasound image from a low-quality image to a high-quality image. As another example, the explaining model may indicate that certain anatomical structures missing from an ultrasound image would transform the ultrasound image from a low-quality image to a high-quality image if present. The user may know how to reposition the ultrasound imaging device to show or not show the anatomical structures in collected ultrasound images and thereby collect a high-quality image. The explaining model may thereby help a user to better use the ultrasound imaging device.

Conversely, if a user views a collected ultrasound image and is unsure why the classification model is classifying the ultrasound image as a high-quality image (e.g., the ultrasound image appears to the user to be low quality), the user may be able to see, from a generated ultrasound image, what changes to the collected ultrasound image may cause the collected ultrasound image to be classified as low quality. The user may thereby gain insight into why the currently collected image was classified as high quality.

As another example, a classification model may classify a collected ultrasound image as showing a particular anatomical view (e.g., an apical two-chamber view of the heart). A user may select an option to generate another ultrasound image, which may be similar to the collected ultrasound image but classified as showing a different anatomical view (e.g., an apical four-chamber view of the heart). As described above, the explaining model may generate the ultrasound image such that changes from the collected ultrasound image to the generated ultrasound image are visually perceptible. Accordingly, if a user views the collected ultrasound image and is unsure why the classification model is classifying the ultrasound image as showing a particular anatomical view rather than another anatomical view, the user may be able to see, from the generated ultrasound image, what changes to the collected ultrasound image may cause it to be classified as showing the other anatomical view. The user may thereby gain insight into how to alter the current ultrasound image collection to collect an ultrasound image showing the other anatomical view. For example, the explaining model may indicate that certain anatomical structures, if removed from an ultrasound image, would transform the ultrasound image from a showing one anatomical view to showing another anatomical view. As another example, the explaining model may indicate that certain anatomical structures missing from an ultrasound image would transform the ultrasound image from showing one anatomical view to showing another anatomical view. The user may know how to reposition the ultrasound imaging device to show or not show the anatomical structures in collected ultrasound images and thereby collect the other anatomical view.

It should be appreciated that the embodiments described herein may be implemented in any of numerous ways. Examples of specific embodiments are provided below for illustrative purposes only. It should be appreciated that the embodiments provided above and below may be used individually, all together, or in any combination of two or more, as aspects of the technology described herein are not limited in this respect.

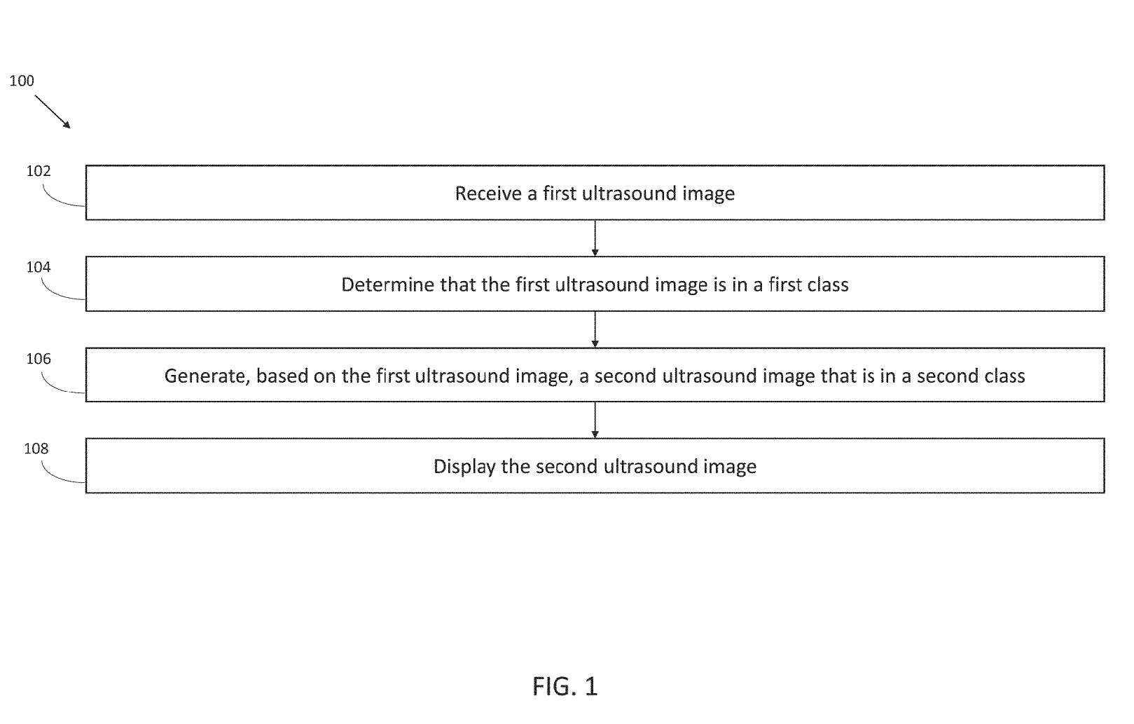

FIG. 1 illustrates an example process 100 for guiding collection of ultrasound data, in accordance with certain embodiments described herein. The process 100 may be performed by a processing device in an ultrasound system. The processing device may be, for example, a mobile phone, tablet, laptop, or server, and may be in operative communication with an ultrasound device.

In act 102, the processing device receives a first ultrasound image. In some embodiments, the ultrasound device may collect raw acoustical data, transmit the raw acoustical data to the processing device, and the processing device may generate the first ultrasound image from the raw acoustical data. In some embodiments, the ultrasound device may collect raw acoustical data, generate scan lines from the raw acoustical data, and transmit the scan lines to the processing device. In such embodiments, the processing device may then generate the first ultrasound image from the scan lines. In some embodiments, the ultrasound device may collect raw acoustical data, generate the first ultrasound image from the raw acoustical data, and transmit the first ultrasound image to the processing device. The ultrasound device may transmit data over a wired communication link (e.g., over Ethernet, a Universal Serial Bus (USB) cable or a Lightning cable) or over a wireless communication link (e.g., over a BLUETOOTH, WiFi, or ZIGBEE wireless communication link) to the processing device, and may transmit data in real-time (i.e., as the data is collected). In some embodiments, the processing device may retrieve the first ultrasound image from memory. The process proceeds from act 102 to act 104.

In act 104, the processing device determines that a classification model classifies the first ultrasound image received in act 102 as belonging to a first class. The first class may be one of multiple classes (e.g., two classes) into which the classification model is trained to classify ultrasound images. For example, the processing device may input the first ultrasound image to a classification model trained to classify ultrasound images into a high-quality class or a low-quality class, and in determining that the first ultrasound image is in a first class, the processing device may determine that the first ultrasound image is in either the low-quality class or the high-quality class. In such embodiments, the classification model may be trained to accept an ultrasound image as an input and estimate a probability (between 0 and 1) that a medical professional would use the image for clinical use, such as for measuring ejection fraction (referred to for simplicity as “clinical use metric”). To train the classification model to estimate this probability, the classification model may be trained with ultrasound images labeled with an indication of whether a medical professional would use the images for clinical evaluation or not. In some embodiments, the classification model may be trained to accept an ultrasound image as an input and to perform some type of segmentation of the image. Furthermore, the classification model may output a confidence metric (between 0 and 1) that the segmentation is correct (referred to for simplicity as “segmentation metric”). The segmentation may be, for example, landmark localization in ultrasound images acquired from the parasternal long axis view of the heart, or left ventricle segmentation (i.e., determining foreground vs. background) in scans acquired from the apical four chamber view of the heart. To train the classification model to perform segmentation on images, the classification model may be trained with images that have been manually segmented. In some embodiments, the classification model may output both a clinical use metric and a segmentation metric. In such embodiments, the method may include calculating a quality metric of the inputted image as the geometric mean of the clinical use metric and the segmentation metric, where the quality metric may range from 0 to 1. Using a geometric mean may help to ensure that the calculated quality metric is not high if either of the clinical use or segmentation metrics is low. The classification model may classify ultrasound images having a quality metric that is from 0 to a threshold value to be in a low-quality class, and the classification model may classify ultrasound images having a quality metric that is from the threshold value to 1 to be in a high-quality class. (Some embodiments may classify ultrasound images having exactly the threshold value to be in the low-quality class, while other embodiments may classify ultrasound images having exactly the threshold value to be in the high-quality class). The threshold value may be, for example, 0.3, 0.4, 0.5, 0.6, 0.7, 0.8, or 0.9.

As another example, the processing device may input the first ultrasound image to a classification model trained to classify ultrasound images as showing a particular anatomical view vs. another anatomical view (e.g., apical two-chamber view of the heart vs. apical four-chamber view of the heart). In determining that the first ultrasound image is in a first class, the processing device may determine that the first ultrasound image shows a particular anatomical view rather than another anatomical view. In such embodiments, the classification model may be trained to accept an ultrasound image as an input and estimate the probability (between 0 and 1) that the ultrasound image shows a particular anatomical view vs. another anatomical view. To train the classification model to estimate this probability, the classification model may be trained with ultrasound images labeled with the anatomical view that the ultrasound image. The classification model may classify ultrasound images having a probability that is from 0 to a threshold value as showing one anatomical view, and the classification model may classify ultrasound images having a probability that is from the threshold value to 1 as showing the other anatomical view. (Some embodiments may classify ultrasound images having exactly the threshold value to show one anatomical view while other embodiments may classify ultrasound images having exactly the threshold value to show the other anatomical view.) The threshold value may be, for example, 0.3, 0.4, 0.5, 0.6, 0.7, 0.8, or 0.9.

In some embodiments, the classification model may be a binary classification model that classifies the first ultrasound image as being in one of two classes (e.g., a high-quality class vs. a low-quality class, or a particular anatomical view vs. another anatomical view). The classification model may be a convolutional neural network, a fully connected neural network, a random forest, a support vector machine, a linear classifier, or any other type of model. The process proceeds from act 104 to act 106.

In act 106, the processing device generates, based on the first ultrasound image received in act 102, a second ultrasound image that the same classification model described with reference to act 104 would classify as belonging to a second class, where the second class is different from the first class. For example, if the classification model classifies the first image received in act 102 as belonging to the first class (e.g., a low-quality class), the second ultrasound image generated in act 106 would be classified by the same classification model as belonging to the second class (e.g., a high-quality class). Similarly, if the first class is a high-quality class, the second class may be a low-quality class. As another example, if the first class is one of two anatomical views classified by the classification model, the second class may be the other anatomical view. In general, if a classification model is a binary classification model that classifies ultrasound images as either the first class or a second class, the processing device may generate at act 106 the second ultrasound image to be in the opposite class as the class of the first ultrasound image received in act 102. In some embodiments, the processing device may generate the second ultrasound image in response to receiving an input from a user. For example, a graphical user interface (GUI) on the processing device may include an option (e.g., a button) that a user can select (e.g., my clicking a button or touching) that triggers generation of the second ultrasound image.

To generate the second ultrasound image, during act 106, the processing device may input the first ultrasound image received in act 102 to an explaining model. The explaining model may be trained to generate the second ultrasound image by introducing changes into one or more portions of the first ultrasound image received in act 102 according to three features. 1. The explaining model may be configured transform the first ultrasound image received in act 102 to the second ultrasound image generated in act 106 in a manner that is detectable by the human eye. 2. The explaining model may be configured to generate the second ultrasound image in act 106 by introducing changes into the first ultrasound image received in act 102 that are spatially localized. Such sparse changes may be more easily interpretable by a human as fewer elements change. 3. The explaining model may be configured to operate consistently with the classification model. The explaining model may operate consistently with the classification model when the classification model predicts different classes for the first ultrasound image inputted to the explaining model and the second ultrasound image generated by the explaining model. These properties of the explaining model may be optimizing certain losses during training of the explaining model, as will be described hereinafter.

In some embodiments, the explaining model may be a convolutional neural network, a fully connected neural network, a random forest, a support vector machine, a linear classifier, or any other type of model.

In addition to the goal of generating a second ultrasound image that is similar to the first ultrasound image, except for a visually perceptible difference, such that the classification model assigns a different class to the second ultrasound image than the first ultrasound image, a goal of the explaining model may also be to generate a binary mask image. The binary mask image may indicate which pixels from the first ultrasound image were changed in order to produce the second ultrasound image. In particular, the binary mask image may be the same size (in pixels) as the first ultrasound image and the second ultrasound image. The value of the pixel at a particular location in the binary mask image may indicate whether the pixel at that same location in the first ultrasound image has been changed or not in order to produce the pixel at that same location in the second ultrasound image. Thus, the explaining model may be configured to illustrate where, via the binary mask image, and how, via the second ultrasound image, the change of the first ultrasound image from classification in the first class to the second class occurs.

More formally, given a binary classification model F(x)∈{0,1}, namely a binary classification model that accepts a first ultrasound image x and outputs a classification in either class 0 or 1, the goal of the explaining model may be to determine a second ultrasound image t and a mask image m such that:

F(x)≠F(t);

x└m≠t└m; and

x└−m≠t└−m, where └ indicates pixel-wise multiplication.

The first equation indicates that the explaining model classifies the first ultrasound image x as a different class than the second ultrasound image t. The second equation indicates that the first ultrasound image x and the second ultrasound image t differ in pixels whose values in the mask image m are 1. The third equation indicates that the first ultrasound image x and the second ultrasound image t match in pixels whose values in the mask image m are 0. It should be appreciated that while the explaining model may be trained according to the above goals, the explaining model may not ultimately meet the goals exactly. For example, the mask image generated by the explaining model may not be exactly binary. The process 100 proceeds from act 106 to act 108.

In act 108, the processing device displays the second ultrasound image. For example, the processing device may display the second ultrasound image on a display screen on the processing device. In some embodiments, the processing device may display the first ultrasound image simultaneously with the second ultrasound image. In some embodiments, the processing device may also display the mask image. In some embodiments, the processing device may highlight, on either or both of the first ultrasound image and the second ultrasound image, pixels corresponding to pixels on the mask image having values exceeding a threshold value (e.g., 0.75, 0.8, 0.85, 0.9, 0.95).

As described above, in some embodiments the first ultrasound image may be classified in a low-quality class. In such embodiments, the processing device may display an indicator of the quality of the first ultrasound image. For example, the processing device may display the indicator as the first ultrasound image is received from an ultrasound device. In some embodiments, a user of the ultrasound device may select an option to generate the second ultrasound image, which may be similar to the first ultrasound image but classified as in a high-quality class. As described above, the explaining model may generate the second ultrasound image such that changes from the first ultrasound image to the second ultrasound image are visually perceptible. Accordingly, if a user views the first ultrasound image and is unsure why the processing device classifies the first ultrasound image as a low-quality image, the user may be able to see, from the second ultrasound image, what changes to the first ultrasound image may cause the first ultrasound image to be classified in the high-quality class. The user may thereby gain insight into how to alter the current ultrasound image collection to collect a high-quality image. For example, the explaining model may indicate that certain anatomical structures, if removed from an ultrasound image, would transform the ultrasound image from a low-quality image to a high-quality image. As another example, the explaining model may indicate that certain anatomical structures missing from an ultrasound image would transform the ultrasound image from a low-quality image to a high-quality image if present. The user may know how to reposition the ultrasound imaging device to show or not show the anatomical structures in collected ultrasound images and thereby collect a high-quality image.

As described above, in some embodiments the first ultrasound image may be classified in a high-quality class. In such embodiments, the processing device may display an indicator of the quality of the first ultrasound image. For example, the processing device may display the indicator as the first ultrasound image is received from an ultrasound device. In some embodiments, a user of the ultrasound device may select an option to generate the second ultrasound image, which may be similar to the first ultrasound image but classified as in a low-quality class. As described above, the explaining model may generate the second ultrasound image such that changes from the first ultrasound image to the second ultrasound image are visually perceptible. Accordingly, if a user views the first ultrasound image and is unsure why the processing device is classifying the first ultrasound image as a high-quality image (e.g., the first ultrasound image appears to the user to be low quality), the user may be able to see, from the second ultrasound image, what changes to the first ultrasound image may cause the first ultrasound image to be classified in the low-quality class. The user may thereby gain insight into why the currently collected image was classified as high quality.

As described above, in some embodiments the first ultrasound image may be classified as showing a particular anatomical view (e.g., an apical two-chamber view of the heart). In such embodiments, the processing device may display an indicator of the anatomical view. For example, the processing device may display the indicator as the first ultrasound image is received from an ultrasound device. In some embodiments, a user of the ultrasound device may select an option to generate the second ultrasound image, which may be similar to the first ultrasound image but classified as showing a different anatomical view (e.g., an apical four-chamber view of the heart). As described above, the explaining model may generate the second ultrasound image such that changes from the first ultrasound image to the second ultrasound image are visually perceptible. Accordingly, if a user views the first ultrasound image and is unsure why the processing device is classifying the first ultrasound image as showing a particular anatomical view rather than another anatomical view, the user may be able to see, from the second ultrasound image, what changes to the first ultrasound image may cause the first ultrasound image to be classified as showing the other anatomical view. The user may thereby gain insight into how to alter the current ultrasound image collection to collect an ultrasound image showing the other anatomical view. For example, the explaining model may indicate that certain anatomical structures, if removed from an ultrasound image, would transform the ultrasound image from a showing one anatomical view to showing another anatomical view. As another example, the explaining model may indicate that certain anatomical structures missing from an ultrasound image would transform the ultrasound image from showing one anatomical view to showing another anatomical view. The user may know how to reposition the ultrasound imaging device to show or not show the anatomical structures in collected ultrasound images and thereby collect the other anatomical view.

Various inventive concepts may be embodied as one or more processes, of which examples have been provided. The acts performed as part of each process may be ordered in any suitable way. Thus, embodiments may be constructed in which acts are performed in an order different than illustrated, which may include performing some acts simultaneously, even though shown as sequential acts in illustrative embodiments. Further, one or more of the processes may be combined and/or omitted, and one or more of the processes may include additional steps.

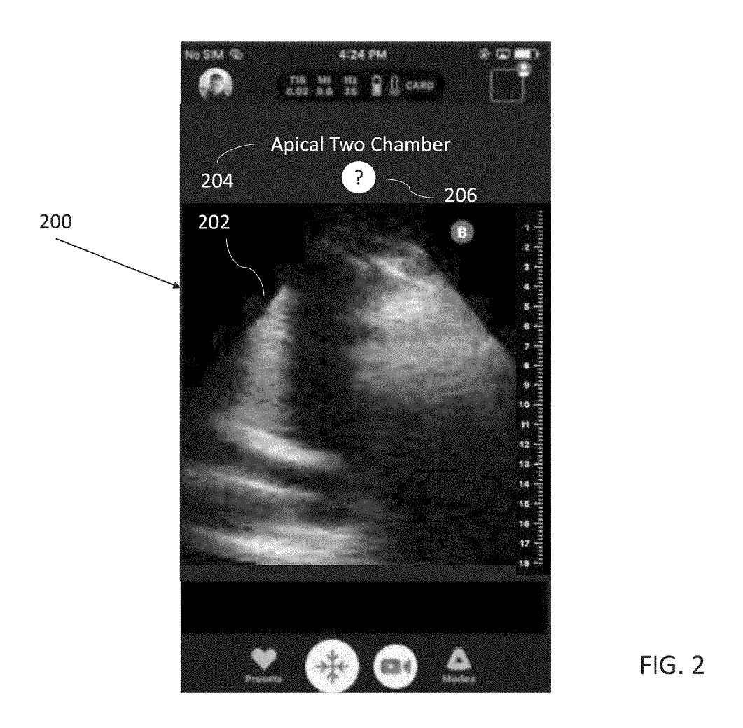

FIG. 2 illustrates an example graphical user interface (GUI) 200 that may be displayed on a display screen of a processing device in an ultrasound system, in accordance with certain embodiments described herein. The GUI 200 generally shows a collected ultrasound image 202 and a classification of the ultrasound image 202 based on an anatomical view shown in the ultrasound image 202. The processing device may be, for example, a mobile phone, tablet, laptop, or server, and may be in operative communication with the ultrasound device. The GUI 200 includes the ultrasound image 202, a class indicator 204, and a button 206.

The ultrasound image 202 may be generated from ultrasound data collected by an ultrasound device. For example, the ultrasound device may transmit ultrasound data (e.g., raw acoustical data or scan lines) to the processing device in real-time as the ultrasound data is collected, and the processing device may generate the ultrasound image 202 from the received ultrasound data and display the ultrasound image 202 on the GUI 200 in real-time. As another example, the ultrasound device may generate the ultrasound image 202 from collected ultrasound data, transmit the ultrasound image 202 to the processing device in real-time, and the processing device may display the ultrasound image 202 in real-time on the GUI 200. In some embodiments, the processing device may retrieve the ultrasound image 202 from memory and display the ultrasound image 202 on the GUI 200. Further description of receiving the ultrasound image 202 may be found with reference to act 102.

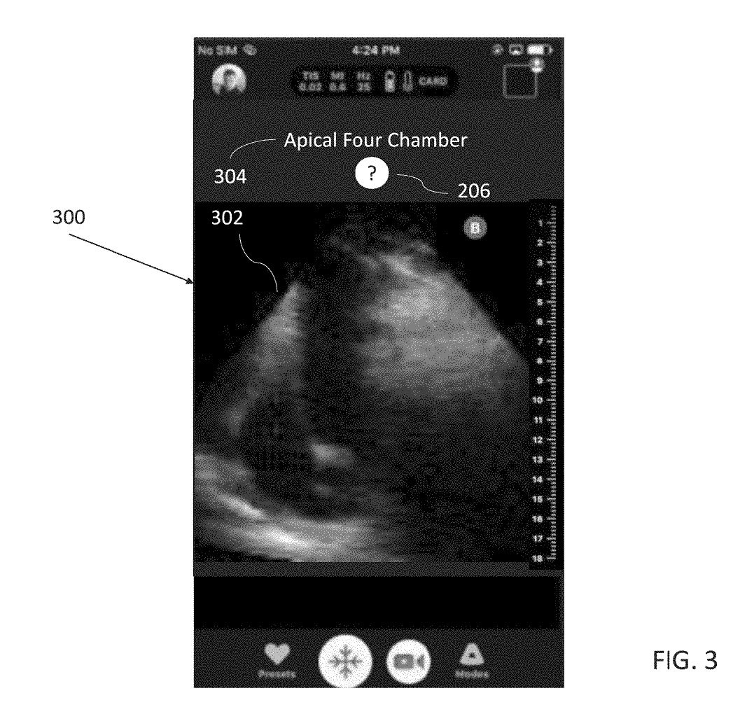

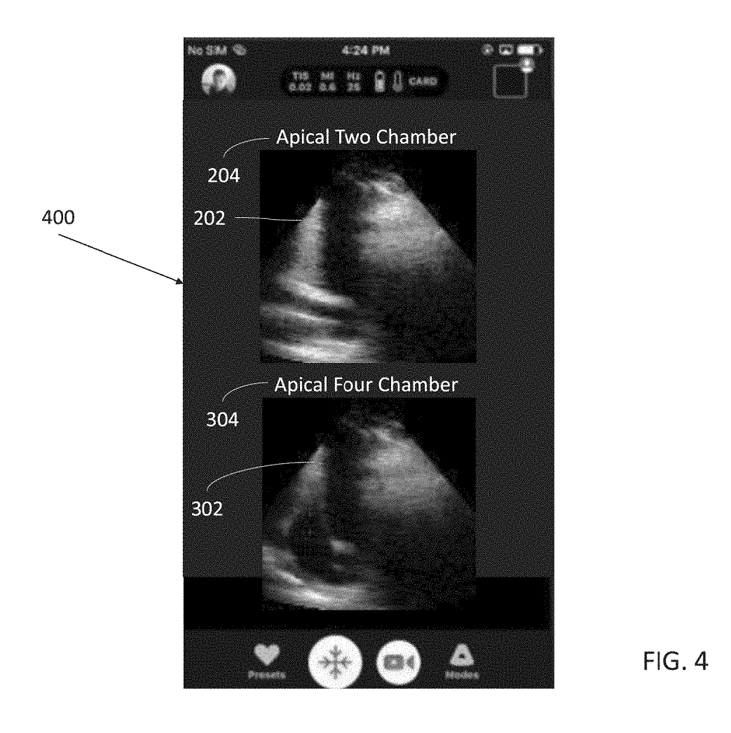

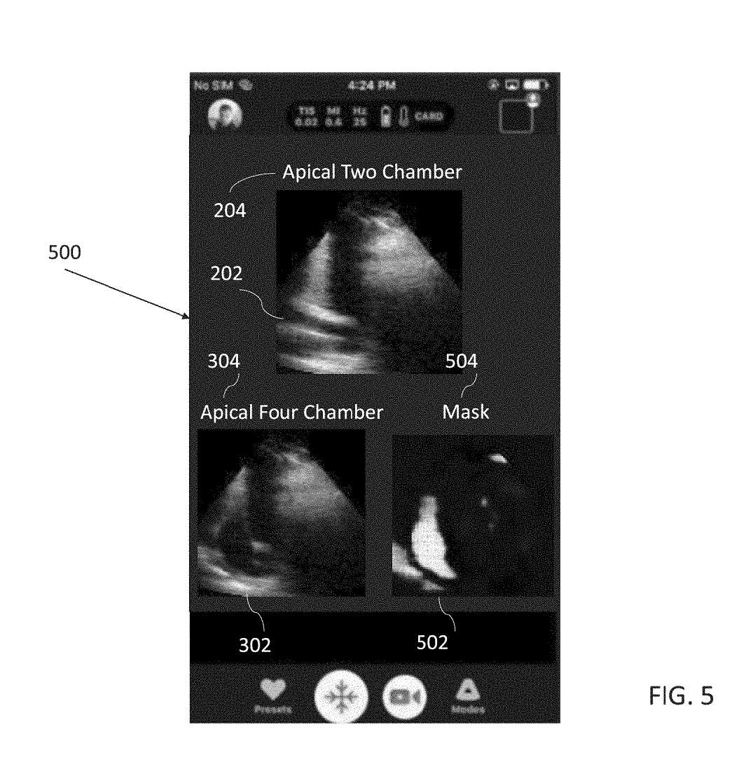

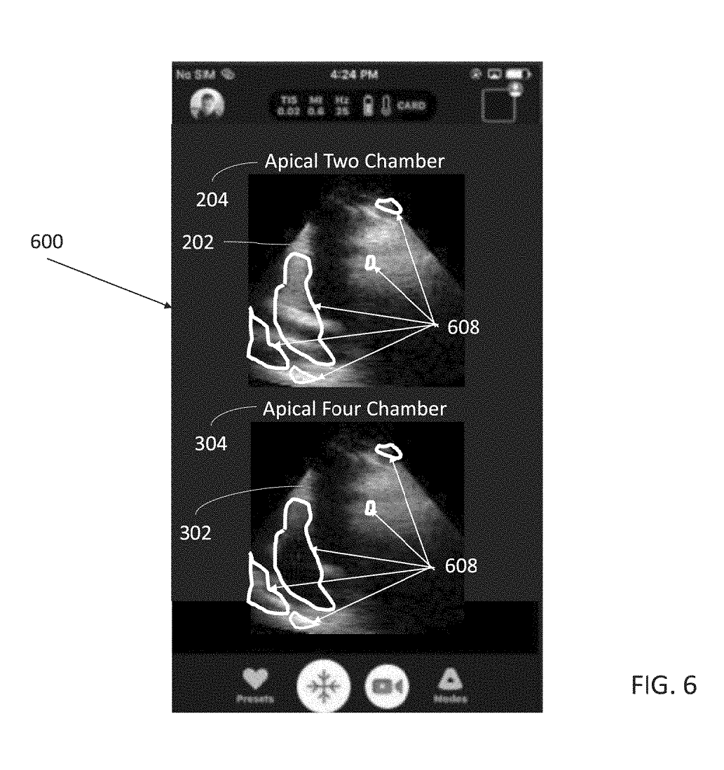

The class indicator 204 may be an indicator of a class in which the ultrasound image 202 is classified. To determine the class, the processing device may input the ultrasound image 202 to a classification model configured to classify the ultrasound image 202. In the example of FIG. 2, the classification model has classified the ultrasound image 202 as showing an apical two-chamber view of the heart, as indicated by the class indicator 204. Further description of determining a class for the ultrasound image 202 may be found with reference to act 104. The button 206 may be an option that a user may select, for example by clicking or touching. In response to selection of the button 206, the GUI 300 shown in FIG. 3, the GUI 400 shown in FIG. 4, the GUI 500 shown in FIG. 5, or the GUI 600 shown in FIG. 6 may be displayed.

FIG. 3 illustrates an example graphical user interface 300 that may be displayed on a display screen of a processing device in an ultrasound system, in accordance with certain embodiments described herein. The GUI 300 may be shown in response to selection of the button 206 from the GUI 200. The GUI 300 generally shows an ultrasound image 302 generated based on the ultrasound image 202 and a classification of the ultrasound image 302 based on an anatomical view shown in the ultrasound image 302. The GUI 300 includes an ultrasound image 302, a class indicator 304, and the button 206. The ultrasound image 302 may be generated by an explaining model based on the ultrasound image 202 collected by an ultrasound device. (As referred to herein, collecting an ultrasound image with an ultrasound device should be understood to mean collecting ultrasound data with the ultrasound device from which the ultrasound image can be generated.) The explaining model may generate the ultrasound image 302 such that the ultrasound image 302 would be classified by the classification model as a different class from the ultrasound image 202. For example, if the classification model is a binary classification that classifies ultrasound images in one of two classes, the classification model may classify the ultrasound 302 generated by the explaining model as the opposite class as the ultrasound image 202. In the example of FIG. 3, the classification model may classify ultrasound images as showing either an apical two-chamber view of the heart or an apical four-chamber view of the heart. Given that the classification model classified the ultrasound image 202 as showing the apical two-chamber view of the heart, the explaining model has generated the ultrasound image 302 such that the classification model may classify the ultrasound 302 as showing an apical four-chamber view of the heart, as indicated by the class indicator 304. The explaining model may generate the ultrasound image 302 such that the ultrasound image 302 differs from the ultrasound image 202 in a manner that is visually perceptible to a human. In response to selection of the button 206, the GUI 200 shown in FIG. 2 may be displayed. Thus, selecting the button 206 may allow a user to switch between viewing the ultrasound image 202 that was collected by the ultrasound device and viewing the ultrasound 302 that was generated by the explaining model based on the ultrasound image 202. This may allow a user to compare the ultrasound image 202 and the ultrasound image 302 and gain insight into why the ultrasound image 202 was classified as showing the apical two-chamber view of the heart rather than the apical four-chamber view of the heart. Further description of generating the ultrasound image 302 may be found with reference to act 106.

FIG. 4 illustrates an example graphical user interface 400 that may be displayed on a display screen of a processing device in an ultrasound system, in accordance with certain embodiments described herein. The GUI 400 may be shown in response to selection of the button 206 from the GUI 200. The GUI 400 includes the ultrasound image 202, the class indicator 204, the ultrasound image 302, and the class indicator 304. By showing the ultrasound image 202 and the ultrasound image 302 simultaneously, a user may be able to compare the ultrasound image 202 and the ultrasound image 302 and gain insight into why the ultrasound image 202 was classified as showing the apical two-chamber view of the heart rather than the apical four-chamber view of the heart.

FIG. 5 illustrates an example graphical user interface 500 that may be displayed on a display screen of a processing device in an ultrasound system, in accordance with certain embodiments described herein. The GUI 500 may be shown in response to selection of the button 206 from the GUI 200. The GUI 500 differs from the GUI 400 in that the GUI 500 includes a mask image 502 and a mask indicator 504. As described above, the explaining model may generate the mask image 502. The mask image 502 may indicate which pixels from the ultrasound image 202 were changed in order to produce the ultrasound image 302. The degree to which pixels in the ultrasound image 202 are changed may be proportional to how close values of pixels at corresponding locations in the mask image 502 are to 1. In other words, pixels in the ultrasound image 202 at locations corresponding to pixels in the mask image 502 that have values closer to 1 (i.e., closer to white) may be substantially changed, while pixels in the ultrasound image 202 at locations corresponding to pixels in the mask image 502 that have values closer to 0 (i.e., closer to black) may not be substantially changed. Thus, the user may gain insight from the mask image 502 regarding which regions of the ultrasound image 202 were changed to change classification of the ultrasound image 202 from showing the apical two-chamber view of the heart to showing the apical four-chamber view of the heart. The mask indicator 504 indicates that the mask image 502 is a mask image.

FIG. 6 illustrates an example graphical user interface 600 that may be displayed on a display screen of a processing device in an ultrasound system, in accordance with certain embodiments described herein. The GUI 600 may be shown in response to selection of the button 206 from the GUI 200. The GUI 600 differs from the GUI 400 in that the GUI 500 includes mask outlines 608 superimposed on the ultrasound image 202 and the ultrasound image 302. The mask outlines 608 may be outlines highlighting regions of the mask image 502 containing pixels having values that are above a certain threshold (e.g., 0.75, 0.8, 0.85, 0.9, 0.95). To generate the mask outlines, edge detection techniques applied to the mask image 502 may be used. Thus, the user may gain insight directly from the mask outlines 608 on the ultrasound image 202 and the ultrasound image 302 regarding which regions of the ultrasound image 202 were changed to change classification of the ultrasound image 202 from showing the apical two-chamber view of the heart to showing the apical four-chamber view of the heart. In some embodiments, the outlines 608 may only be shown on the ultrasound image 202 or only on the ultrasound image 302. Further description of displaying the ultrasound image 302 may be found with reference to act 108. It should be appreciated that while the example anatomical views in FIGS. 2-6 are the apical two-chamber view of the heart and the apical four-chamber view of the heart, other anatomical views and other anatomical structures may be used.

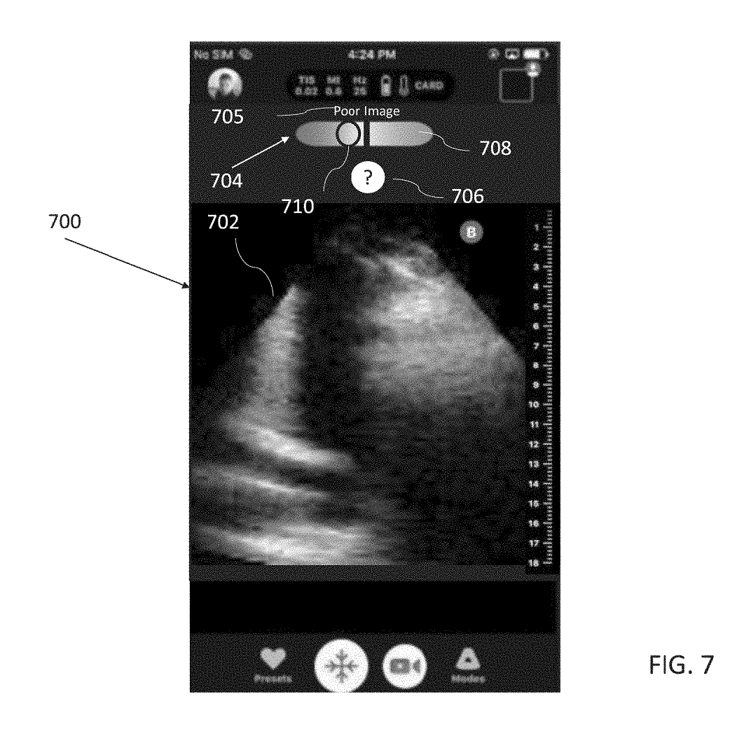

FIG. 7 illustrates an example graphical user interface 700 that may be displayed on a display screen of a processing device in an ultrasound system, in accordance with certain embodiments described herein. The GUI 700 generally shows a collected ultrasound image 702 and a classification of the ultrasound image 702 based on the quality of the ultrasound image 202. The processing device may be, for example, a mobile phone, tablet, laptop, or server, and may be in operative communication with the ultrasound device. The GUI 700 includes an ultrasound image 702, a graphical quality indicator 704, a textual quality indicator 705, and a button 706. The graphical quality indicator 704 includes a bar 708 and a marker 710.

The ultrasound image 702 may be generated from ultrasound data collected by an ultrasound device. For example, the ultrasound device may transmit ultrasound data (e.g., raw acoustical data or scan lines) to the processing device in real-time as the ultrasound data is collected, and the processing device may generate the ultrasound image 702 from the received ultrasound data and display the ultrasound image 702 on the GUI 700 in real-time. As another example, the ultrasound device may generate the ultrasound image 702 from collected ultrasound data, transmit the ultrasound image 702 to the processing device in real-time, and the processing device may display the ultrasound image 702 in real-time on the GUI 700. In some embodiments, the processing device may retrieve the ultrasound image 702 from memory and display the ultrasound image 702 on the GUI 700. Further description of receiving the ultrasound image 702 may be found with reference to act 102.

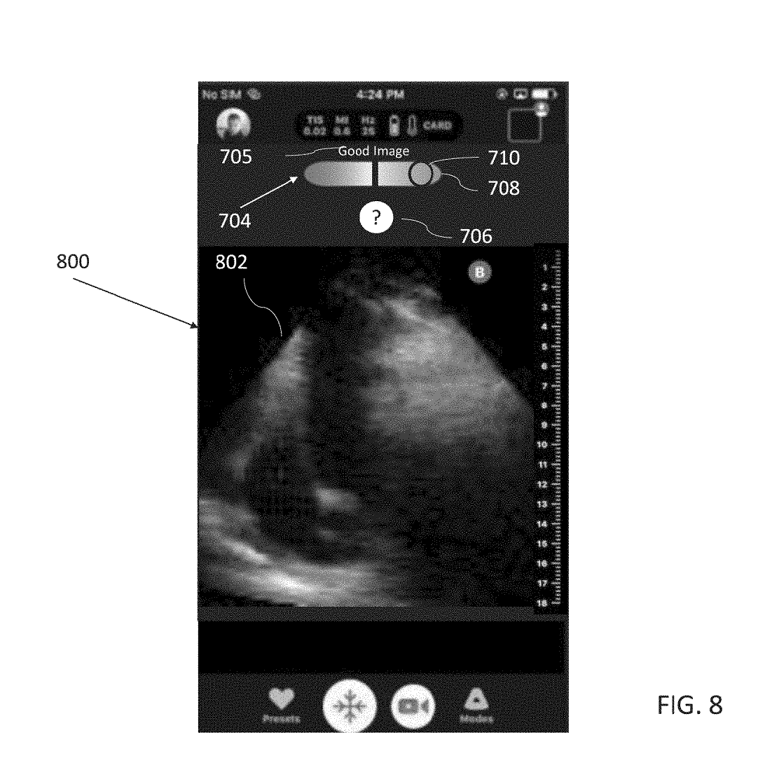

The graphical quality indicator 704 may indicate a quality metric determined for the ultrasound image 702. To determine the quality metric, the processing device may be configured to input the ultrasound image 702 to a classification model trained to determine the quality metric for the ultrasound image 702. The quality metric may range from 0 to 1. The graphical quality indicator 704 may display the quality metric by displaying the marker 710 at a particular position relative to the bar 708. In particular, the distance from the left edge of the bar 708 to the center of the marker 710 divided by the distance from the left edge of the bar 708 to the right edge of the bar 708 may be substantially equal to the quality metric. The classification model may classify ultrasound images having a quality metric below a certain threshold as being in a low-quality class and ultrasound images having a quality metric above a certain threshold as being in a high-quality class. For example, the threshold may be 0.3, 0.4, 0.5, 0.6, 0.7, 0.8, or 0.9. The textual quality indicator 705 may indicate this class. In the example of FIG. 7, the classification model has classified the ultrasound image 702 in the low-quality class, as indicated by the textual quality indicator 705, which in the example illustrated indicates “Poor Image.” Other textual indicators may also be used, however. Further description of determining a class for the ultrasound image 702 may be found with reference to act 104. The button 706 may be an option that a user may select, for example by clicking or touching. In response to selection of the button 706, the GUI 800 shown in FIG. 8 may be displayed.

FIG. 8 illustrates an example graphical user interface 800 that may be displayed on a display screen of a processing device in an ultrasound system, in accordance with certain embodiments described herein. The GUI 800 differs from the GUI 700 in that the GUI 800 includes, instead of the ultrasound image 702, an ultrasound image 802 generated from the ultrasound image 702. The ultrasound image 802 may be generated by an explaining model based on the ultrasound image 702 that was generated from ultrasound data collected by an ultrasound device. The explaining model may generate the ultrasound image 802 such that the ultrasound image 802 would be classified by the classification model as being in a different class than the ultrasound image 702. For example, if the classification model is a binary classifier that classifies ultrasound images as being in one or two categories, the classification model may classify the ultrasound image 802 generated by the explaining model as being in the opposite class as the ultrasound image 702. In the example of FIG. 8, the classification model may classify ultrasound images as either being in a low-quality class or a high-quality class. Given that the classification model classified the ultrasound image 702 as being in the low-quality class, the explaining model has generated the ultrasound image 802 such that the classification model may classify the ultrasound image 802 as being in the high-quality class, as indicated by the graphical quality indicator 704 and the textual quality indicator 705. The explaining model may generate the ultrasound image 802 such that the ultrasound image 802 differs from the ultrasound image 702 in a manner that is visually perceptible to a human. In response to selection of the button 706, the GUI 700 shown in FIG. 7 may be displayed. Thus, selecting the button 706 may allow a user to switch between viewing the ultrasound image 702 that was generated from ultrasound data collected by an ultrasound device, and viewing the ultrasound image 802 that was generated by the explaining model based on the ultrasound image 702. This may allow a user to compare the ultrasound image 702 and the ultrasound image 802 and gain insight into why the ultrasound image 702 was classified as being low quality. Further description of generating the ultrasound image 802 may be found with reference to act 106. It should be appreciated that any of the GUI embodiments shown in FIGS. 2-6 for the example of anatomical view classification may be applied to the example of quality classification. For example, a collected ultrasound image classified in a low-quality class may be shown simultaneously with an ultrasound image generated by an explaining mode to be in a high-quality class, or a collected ultrasound image classified in a high-quality class may be shown simultaneously with an ultrasound image generated by an explaining mode to be in a low-quality class. Additionally, a mask image may be shown simultaneously with one or more of a collected ultrasound image and a generated ultrasound image, and/or outlines derived from a mask image may be superimposed on one or more of a collected ultrasound image and a generated ultrasound image.

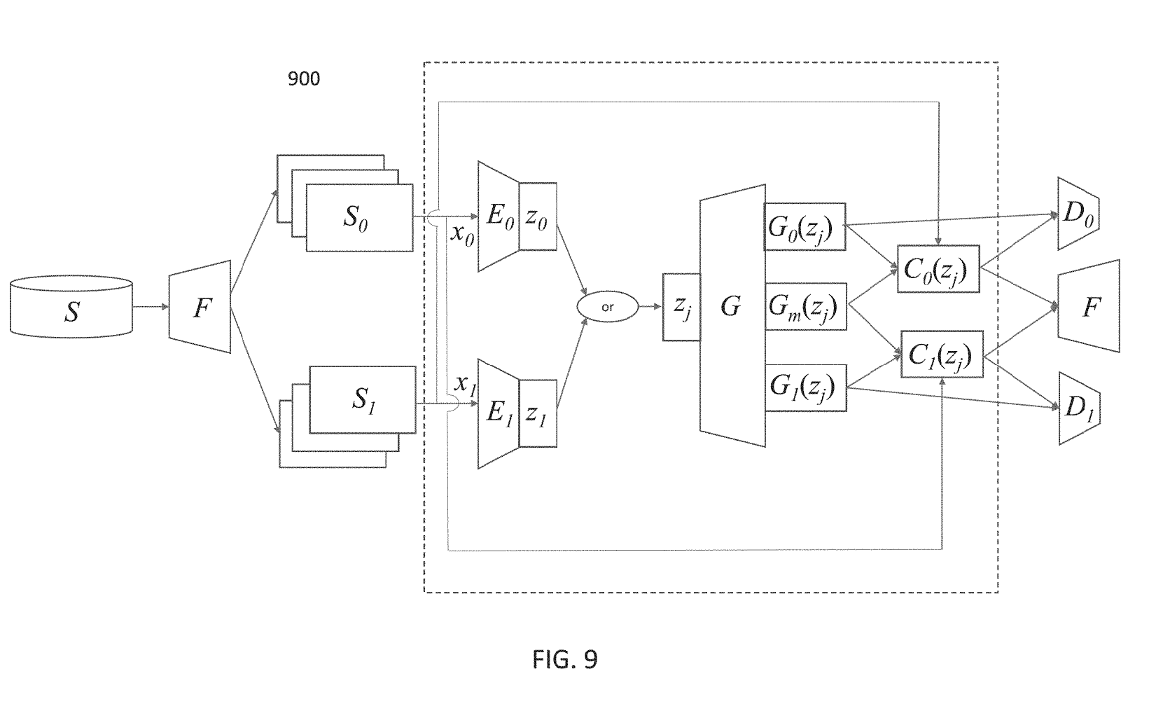

FIG. 9 illustrates an example architecture for an explaining model 900 in accordance with certain embodiments described herein. The explaining model 900 may be, for example, the explaining model used in the process 100 for generating the second ultrasound image, for generating the ultrasound image 302 in FIGS. 3-6, and/or for generating the ultrasound image 802 in FIG. 8. The explaining model 900 includes an encoder E₀, an encoder E₁, and a generator G. The explaining model 900 is configured to explain the output of a classification model F. The classification model F is configured to classify an input image from a dataset of images S as either being of a class 0 or 1, where the images from S that are classified as class 0 are referred to as S₀ and images from S that are classified as class 1 are referred to as S₁. For example, class 0 may be a low-quality class and class 1 may be a high-quality class. As another example, class 0 may be an apical two-chamber view class and class 1 may be an apical four-chamber view class. To classify an input image, F may output a probability that the input image is of class 0. The probability may be proportional to the confidence that the input image is in class 0 vs. class 1. In other words, a probability closer to 1 may indicate confidence that the input image is in class 0, and a probability closer to 0 may indicate confidence that the input image is in class 1. The explaining model 900 is configured to accept an input image x. For example, the image x may be the ultrasound image received in act 102, the ultrasound image 202, and/or the ultrasound image 702. If the image x is in S₀, then the explaining model 900 inputs x to the encoder E₀. If the image x is in S₁, then the explaining model 900 inputs the image x to the encoder E₁.

The encoder E₀ is configured to encode the image x as a hidden vector z₀ and the encoder E₁ is configured to encode the image x as a hidden vector z₁. The hidden vectors z₀ and z₁ may be representations of the image x that are smaller in size than the image x. The explaining model 900 inputs either the hidden vector z₀ or the hidden vector z₁ to the generator G. Henceforth, the image x will be referred to as xⱼ, where j=0 if x is in S₀ and j=1 if x is in S₁. In general:

x=xⱼ, j∈{0,1}, x∈Sⱼ

Additionally, the hidden vector inputted to the generator G will be referred to as zⱼ. In general:

zⱼ=Eⱼ(xⱼ), j∈{0,1}, x∈Sⱼ

The generator G generates, based on zⱼ, a reconstructed image Gⱼ(zⱼ), a transformed image G₁₋ⱼ(zⱼ), and a mask Gₘ(zⱼ). The explaining model 900 outputs a composite image C₁₋ⱼ(zⱼ) based on the reconstructed image G₁₋ⱼ(zⱼ), the mask Gₘ(zⱼ), and the image xⱼ. As will be described hereinafter, the generator G may be trained such that the reconstructed image Gⱼ (zⱼ) is in class j (in particular, that the classification model F would classify Gⱼ(zⱼ) as being in class j), and such that G₁₋ⱼ(zⱼ) is in class 1-j (in particular, that the classification model F would classify G₁₋ⱼ(zⱼ) as being in class 1-j). The generator G may be further trained such that the mask Gₘ(zⱼ) is a mask indicating certain changes to be made from the image xⱼ when forming the composite image C₁₋ⱼ(zⱼ). In particular, the degree to which pixels in C₁₋ⱼ(zⱼ) have been substantially changed from the values of the corresponding pixels in xⱼ may be proportional to how close the values of corresponding pixels in Gₘ(zⱼ) are to 1. In other words, pixels of Gₘ(zⱼ) that have values closer to 1 may indicate that the values of corresponding pixels in C₁₋ⱼ(zⱼ) have been substantially changed from the values of the corresponding pixels in xⱼ and pixels of Gₘ(zⱼ) that have values closer to 0 may indicate that the values of corresponding pixels in C₁₋ⱼ(zⱼ) have not been substantially changed from the values of the corresponding pixels in xⱼ. The generator G may be trained such that the mask image Gₘ(zⱼ) indicates changes to be made to the image xⱼ that cause the resulting composite image C₁₋ⱼ(zⱼ) to be classified in an opposite class as xⱼ. In some embodiments, the composite image C₁₋ⱼ(zⱼ) may be a weighted sum of xⱼ and G₁₋ⱼ(zⱼ). The weighted sum may be determined by the mask image Gₘ(zⱼ). In particular, the weighting of pixels of G₁₋ⱼ(zⱼ) vs. pixels of xⱼ may be proportional to how close the values of corresponding pixels in Gₘ(zⱼ) are to 1. In particular, pixels of xⱼ may be weighted more in the sum when the corresponding pixels of Gₘ(zⱼ) are closer to 0, and pixels of G₁₋ⱼ(zⱼ) may be weighted more in the sum when the corresponding pixels of Gₘ(zⱼ) are closer to 1. Thus, the composite image C₁₋ⱼ(zⱼ)may be a blend of the reconstructed image G₁₋ⱼ(zⱼ) and the image xⱼ. In particular:

C₁₋ⱼ(xⱼ)=xⱼ⊙(1−Gₘ(zⱼ))+G₁₋ⱼ(zⱼ)⊙Gₘ(zⱼ), where ⊙ represents pixel-wise multiplication. The composite image C₁₋ⱼ(xⱼ) may be ultrasound image generated in act 206, the ultrasound image 302, and/or the ultrasound image 802.

To train the explaining model 900 to produce the reconstructed images Gⱼ(j) and G₁₋ⱼ(zⱼ), the mask Gₘ(zⱼ), and the composite image C₁₋ⱼ(zⱼ), a discriminator D₀ and a discriminator D₁ (shown in FIG. 9) are used. Each of the discriminators D₀ and D₁ is configured to accept an image as an input and output a probability that the input image is real or fake (where fake means generated by G). In some embodiments, the discriminator D₀ is configured to output a probability that an input image of class 0 is real and the discriminator D₀ is configured to output a probability that an input image of class 1 is real. The probability may be proportional to the confidence of the discriminator that the input image is real. In other words, a probability close to 1 may indicate confidence that the input image is real and a probability close to 0 may indicate confidence that the input image is fake. In general, training proceeds to encourage the explaining model 900 to produce reconstructed images G₁₋ⱼ(zⱼ) and composite images C₁₋ⱼ(zⱼ) that appear to be real and that are classified as the opposite class of the input image xⱼ. The explaining model 900 may be considered an adversarial network in that during training, the discriminators D₀ and D₁ and the generator G may modulate their parameters to optimize opposite results such that the discriminators D₀ and D₁ improve their ability to discriminate between real images and fake images generated by the generator G, and the generator G improves its ability to generate fake images such that the discriminators D₀ and D1 are unable to differentiate between fake and real images. Training also proceeds to encourage the mask Gₘ(zⱼ) to exhibit certain characteristics described further hereinafter.

The explaining model 900 is trained by inputting, to the explaining model 900, training images that have been classified by the classification model F, and adjusting parameters of the generator G, the encoders E₀ and E₁, and the discriminators D₀ and D₁ based on the output of the model 900 to optimize an objective. In some embodiments, the objective may be

$${min}_{G , {E}_{0} , {E}_{1}} {max}_{{D}_{0} , {D}_{1}} {ℒ}_{GAN} + {ℒ}_{classifier} + {ℒ}_{recon} + {ℒ}_{prior} .$$

[symbol]

_{GAN}, [symbol] _{classifier}, [symbol] _{recon}, [symbol] ₚᵣᵢₒᵣ may be loss terms, and the parameters of generator G and the encoders E₀ and E₁ may be adjusted to minimize the loss terms [symbol] _{GAN}, [symbol] _{classifier}, [symbol] _{recon}, [symbol] ₚᵣᵢₒᵣ. The parameters of the discriminators D₀ and D₁ may be adjusted to maximize the loss term [symbol] _{GAN} (which, as will be described hereinafter, may be the only loss terms dependent on the discriminators D₀ and D₁).

In some embodiments, [symbol] _{GAN} (where GAN is an abbreviation of generative adversarial network) is a loss term encouraging the explaining model 900 to generate fake images that appear to be real. In particular, [symbol] _{GAN} encourages the adversarial nature of the discriminators D₀ and D₁ and the generator G. [symbol] _{GAN} may be high when the discriminator correctly discriminates between real images and fake images generated by the generator G. This is the objective towards which the parameters of the discriminators D₀ and D₁ are optimized. [symbol] _{GAN} may be low when the discriminators D₀ and D₁ are unable to differentiate between fake and real images. This is the objective towards which the parameters of E₀ and E₁ are optimized. As described above, in some embodiments the discriminator D₀ is configured to output a probability that an input image of class 0 is real and the discriminator D₀ is configured to output a probability that an input image of class 1 is real. The probability may be proportional to the confidence of the discriminator that the image is real. In other words, a probability close to 0 may indicate confidence that the input image is real and a probability close to 0 may indicate confidence that the input image is fake. In such embodiments:

[symbol]

_{GAN}= [symbol] _{GAN:0}+ [symbol] _{GAN:1}, where:

[symbol]

_{GAN:j}= [symbol] _{x∈S}_{ⱼ} log(Dⱼ(x))+ [symbol] _{x∈S}_{ⱼ}[log(1−Dⱼ(Gⱼ(Eⱼ(x))))]+ [symbol] _{x∈S}_{₁₋ⱼ}[log(1−Dⱼ(Gⱼ(E₁₋ⱼ(x))))]+ [symbol] _{x∈S}_{₁₋ⱼ}[log(1−Dⱼ(Cⱼ(E₁₋ⱼ(x))))],

[symbol] _{x∈S}_{ⱼ} indicates the expected value given that the image x is in Sⱼ and [symbol] _{x∈S}_{₁₋ⱼ} indicates the expected value given that the image x is in S₁₋ⱼ.

For a given class j, the first term of [symbol] _{GAN:j} may penalize Eⱼ and G if the discriminator Dⱼ outputs a high probability that a real image of class j is real. The second term of [symbol] _{GAN:j} may penalize Eⱼ and G if the discriminator Dⱼ outputs a low probability that a reconstructed image of class j generated by the generator G based on a real image of class j is real. The third term of [symbol] _{GAN:j} may penalize Eⱼ and G if the discriminator Dⱼ outputs a low probability that a transformed image of class j generated by the generator G based on real image of class 1-j is real. The fourth term of [symbol] _{GAN:j} may penalize Eⱼ and G if the discriminator Dⱼ outputs a low probability that a composite image of class j generated from a reconstructed image of class j, a mask image generated by the generator G, and a real image of class 1-j, is real. Conversely, for a given class j, the first term of [symbol] _{GAN:j} may penalize the discriminator Dⱼ if Dⱼ outputs a low probability that a real image of class j is real. The second term of [symbol] _{GAN:j} may penalize the discriminator Dⱼ if Dⱼ outputs a high probability that a reconstructed image of class j generated by the generator G based on a real image of class j is real. The third term of [symbol] _{GAN:j} may penalize the discriminator Dⱼ if Dⱼ outputs a high probability that a transformed image of class j generated by the generator G based on a real image of class 1-j is real. The fourth term of [symbol] _{GAN:j} may penalize the discriminator Dⱼ if Dⱼ outputs a high probability that a composite image of class j generated from a reconstructed image of class j, a mask image generated by the generator G, and a real image of class 1-j, is real.

In some embodiments, [symbol] _{classifier} is a loss term encouraging the explaining model 900 to output composite images that the classification model F will classify as the intended class. For example, [symbol] _{classifier} may encourage the explaining model 900 to output a C₁(zⱼ) that the classification model F will classify as class 1 and to output a C₀(zⱼ) that the classification model F will classify as class 0. As discussed above, to classify an input image in some embodiments, F may output a probability that the input image is of class 0. The probability may be proportional to the confidence of F that the image is in class 0. In other words, a probability closer to 1 indicates confidence that the input image is of class 0, and a probability closer to 0 indicates confidence that the input image is of class 1. In such embodiments:

[symbol]

_{classifier}= [symbol] _{x∈S}_{₀}[−log(F(C₁(E₀(x))))+ [symbol] _{x∈S}_{₁}[−log(1−F(C₀(E₁(x))))].

The first term of [symbol] _{classifier} may penalize the explaining model 900 if the classification model F classifies C₁(x₀) as class 0. The second term of [symbol] _{classifier} may penalize E₀, E₁, and G if the classification model F classifies C₀(x₁) as class 1.

In some embodiments, [symbol] _{recon} is a loss term encouraging reconstructed images Gⱼ(zⱼ) to be similar to inputted images xⱼ. In some embodiments:

[symbol] _{recon}=Σ_{j∈0,1} [symbol] _{x∈S}_{ⱼ}∥Gⱼ(Eⱼ(x))−x∥², where the double brackets indicate summation of squared pixels across an image.

In some embodiments, [symbol] ₚᵣᵢₒᵣ may encourage the explaining model 900 to output a mask Gₘ(zⱼ) that exhibits certain characteristics. In particular, these characteristics may encourage changes to the input image x that are local to a particular part of the image x and visually perceptible. In some embodiments:

[symbol]

ₚᵣᵢₒᵣ= [symbol] _{consistency}+ [symbol] _{count}+ [symbol] ₛₘₒₒₜₕₙₑₛₛ+ [symbol] _{entropy}.

In some embodiments, [symbol] _{consistency} may ensure that if a pixel is not masked (as indicated by Gₘ(zⱼ), then the transformed image G₁₋ⱼ(zⱼ) has not altered that pixel from the original image xⱼ. In some embodiments:

[symbol] _{consistency}=Σ_{j∈0,1} [symbol] _{x∈S}_{ⱼ}[∥(1−Gₘ(zⱼ))⊙Gⱼ(zⱼ)−(1Gₘ(zⱼ))⊙G₁₋ⱼ(zⱼ)∥²].

The first term inside the double brackets of [symbol] _{consistency} may represent the unmasked pixels of the reconstructed image G₁(z₁) (which due to [symbol] _{recon} should be similar to xⱼ). The second term inside the double brackets of [symbol] _{consistency} may represent the unmasked pixels of the transformed image G₁₋ⱼ(zⱼ). The difference between these two terms may represent the difference between unmasked pixels of the reconstructed image Gⱼ(zⱼ) and unmasked pixels of the transformed image G₁₋ⱼ(zⱼ), and therefore [symbol] _{consistency} may penalize the explaining model 900 if unmasked pixels of the reconstructed image Gⱼ(zⱼ) and unmasked pixels of the transformed image G₁₋ⱼ (zⱼ) are different.

In some embodiments, [symbol] _{count} may encourage the ratio of pixels changed with respect to total pixels from the original image xⱼ to the composite image C₁₋ⱼ(zⱼ) to be less than a certain ratio. In some embodiments:

$${ℒ}_{count } = {\sum }_{j ∈ 0 , 1} {𝔼}_{x ∈ {S}_{j}} [ max ( \frac{1}{n} \vert {G}_{m} ( {z}_{j} ) \vert , κ ) ] ,$$

where n is the number of pixels in Gₘ(zⱼ), κ is the desired ratio, and the single brackets indicate the sum of the absolute values of pixels across an image.

The sum of the absolute values of pixels across Gₘ(zⱼ) may be indicative of the number of pixels in Gₘ(zⱼ) that are close to 1, which are those pixels that are changed from the original image xⱼ to the composite image C₁₋ⱼ(zⱼ). Dividing this number by n, the number of pixels in Gₘ(zⱼ), may be indicative of the ratio of pixels changed with respect to total pixels from the original image xⱼ to the composite image C₁₋ⱼ(zⱼ). Minimizing [symbol] _{count} which is the maximum of

$$\frac{1}{n} \vert {G}_{m} ( {z}_{j} ) \vert$$

and κ, may encourage the ratio of pixels changed with respect to total pixels from the original image xⱼ to the composite image C₁₋ⱼ(zⱼ) to be less than κ.

In some embodiments, [symbol] ₛₘₒₒₜₕₙₑₛₛ may encourage the mask Gₘ(zⱼ) to be localized by penalizing transitions across the mask Gₘ(zⱼ). In some embodiments:

[symbol] ₛₘₒₒₜₕₙₑₛₛ=Σ_{j∈0,1} [symbol] _{x∈S}_{ⱼ}|∇Gₘ(zⱼ)|, where ∇Gₘ(zⱼ) is the total variation of ∇Gₘ(zⱼ). For further description of total variation, see Rudin, Leonid I., Stanley Osher, and Emad Fatemi, “Nonlinear total variation based noise removal algorithms,” Physica D: nonlinear phenomena 60.1-4 (1992): 259-268, which is incorporated by reference herein in its entirety.

In some embodiments, [symbol] _{entropy} may encourage the mask Gₘ(zⱼ) to be as binary as possible. In some embodiments:

[symbol]

_{entropy}=Σ_{j∈0,1} [symbol] _{x∈S}_{ⱼ}Σ_{all pixels}[min(Gₘ(zⱼ), 1−Gₘ(zⱼ))].

[symbol] _{entropy} includes a sum over all pixels of Gₘ(zⱼ) of the minimum, at each pixel of Gₘ(zⱼ), of the pixel value and 1 minus the pixel value. Since Gₘ(zⱼ) ranges from 0 to 1, this minimum value will be as low of possible, namely 0, when pixel values are either 0 or 1.

In some embodiments, the explaining model 900 may be extended to classifiers F that classify an input image as one of more than two classes. In particular, there may be one encoder per class, and the generator may generate a reconstructed image for each class as well as a mask image for each pair of classes.

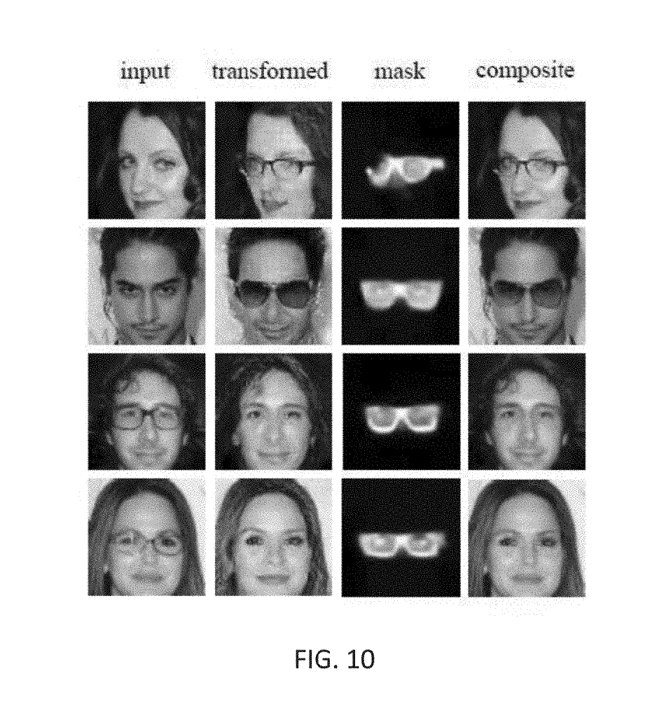

FIG. 10 illustrates example inputs to and outputs from an explaining model (e.g., the explaining model 900) in accordance with certain embodiments described herein. Each row of four images illustrates an input image x to the explaining model 900 (where the image x is of class j), an output transformed image G₁₋ⱼ(zⱼ), an output mask image Gₘ(zⱼ), and an output composite image C₁₋ⱼ(z₁). The input images, transformed images, and output composite images each show a person. The two classes in FIG. 10 are 0. The person is wearing glasses and 1. The person is not wearing glasses. Thus, for an input image where the person is not wearing glasses, the person in the transformed image is wearing glasses. The mask image is localized to the eyeglasses region, and the composite image blends the face of the input image with the eyeglasses region of the transformed image. For an input image where the person is wearing glasses, the person in the transformed image is not wearing glasses. The mask image is localized to the eye region, and the composite image blends the face of the input image with the eye region of the transformed image.

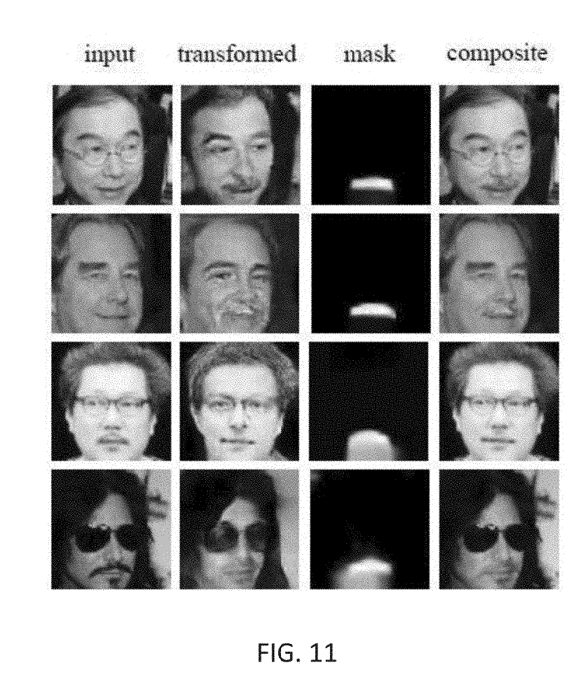

FIG. 11 illustrates example inputs to and outputs from an explaining model (e.g., the explaining model 900) in accordance with certain embodiments described herein. FIG. 11 is similar to FIG. 10, except that the two classes are 0. The person has a mustache and 1. The person does not have a mustache.

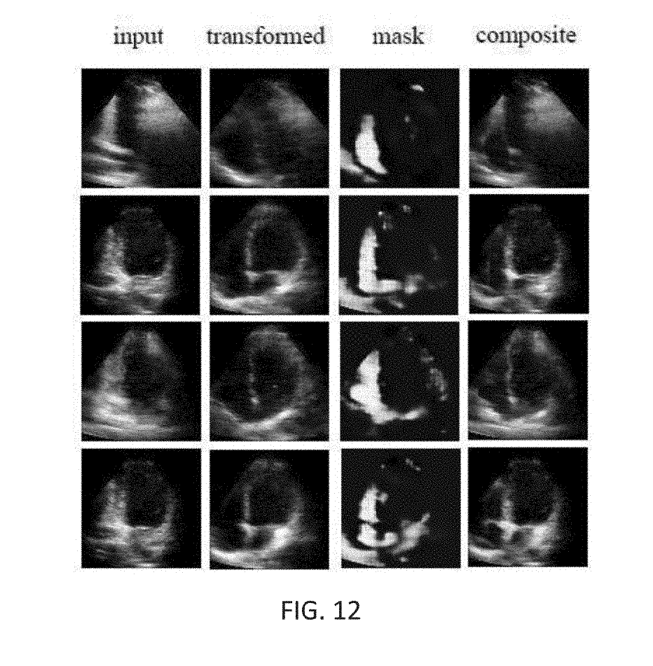

FIG. 12 illustrates example input to and outputs from an explaining model (e.g., the explaining model 900) in accordance with certain embodiments described herein. The input images, transformed images, and output composite images each show ultrasound images. FIG. 12 is similar to FIG. 10, except that the two classes are 1. The ultrasound image shows an apical two-chamber view of the heart and 2. The ultrasound image shows an apical four-chamber view of the heart. All the input images are of the first class and all the composite images are of the second class.

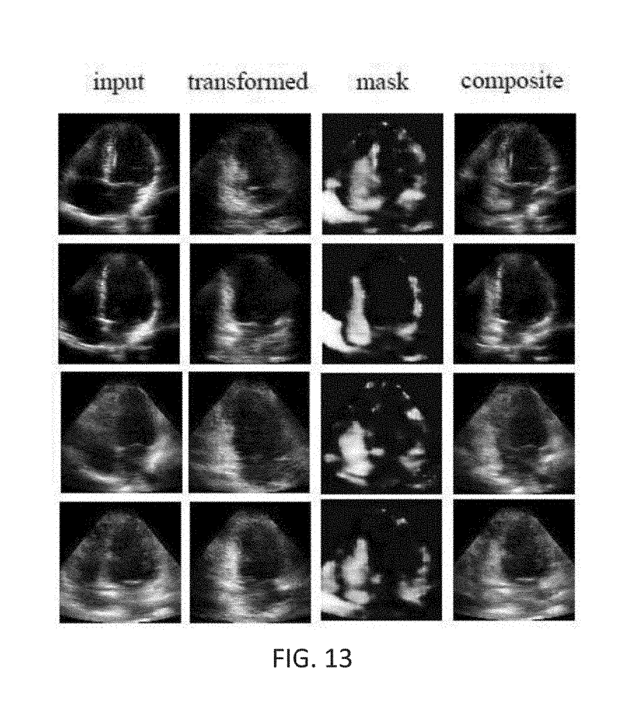

FIG. 13 illustrates example input to and outputs from an explaining model (e.g., the explaining model 900) in accordance with certain embodiments described herein. FIG. 13 is similar to FIG. 12, except that all the input images are in the second class and all the composite images are in the first class.

Various aspects of the present disclosure may be used alone, in combination, or in a variety of arrangements not specifically described in the embodiments described in the foregoing and is therefore not limited in its application to the details and arrangement of components set forth in the foregoing description or illustrated in the drawings. For example, aspects described in one embodiment may be combined in any manner with aspects described in other embodiments.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

As used herein, reference to a numerical value being between two endpoints should be understood to encompass the situation in which the numerical value can assume either of the endpoints. For example, stating that a characteristic has a value between A and B, or between approximately A and B, should be understood to mean that the indicated range is inclusive of the endpoints A and B unless otherwise noted.

The terms “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having described above several aspects of at least one embodiment, it is to be appreciated various alterations, modifications, and improvements will readily occur to those skilled in the art. Such alterations, modifications, and improvements are intended to be object of this disclosure. Accordingly, the foregoing description and drawings are by way of example only.

What is claimed is:

1. A method, comprising:
determining, with a processing device, that a classification model classifies a first ultrasound image as belonging to a first class;
generating, based on the first ultrasound image, a second ultrasound image that the classification model would classify as belonging to a second class, wherein the second class is different from the first class; and
displaying the second ultrasound image.

2. The method of claim 1, wherein generating the second ultrasound image comprises changing one or more portions of the first ultrasound image.

3. The method of claim 1, wherein generating the second ultrasound image comprises inputting the first ultrasound image to an explaining model configured to accept the first ultrasound image as an input and output the second ultrasound image based on the first ultrasound image.

4. The method of claim 1, wherein determining that the classification model classifies the first ultrasound image as belonging to the first class comprises inputting the first ultrasound image to the classification model.

5. The method of claim 4, wherein the classification model is configured to classify the inputted ultrasound image according to a quality of the inputted ultrasound image.

6. The method of claim 4, wherein the classification model is configured to classify the inputted ultrasound image according to an anatomical view shown in the inputted ultrasound image.

7. The method of claim 4, wherein the classification model is configured to classify ultrasound images as belonging to either the first class or the second class.

8. The method of claim 1, wherein the first class comprises a low-quality class and the second class comprises a high-quality class.

9. The method of claim 1, wherein the first class comprises a first anatomical view and the second class comprises a second anatomical view.

10. The method of claim 1, wherein generating the second ultrasound image comprises generating a composite of the first ultrasound image and a transformed version of the first ultrasound image.

11. The method of claim 1, further comprising generating a mask image indicating changes from the first ultrasound image to the second ultrasound image.

12. The method of claim 11, further comprising displaying the mask image.

13. The method of claim 11, further comprising displaying the mask image and the second ultrasound image simultaneously.

14. The method of claim 11, further comprising displaying the mask image, the second ultrasound image, and the first ultrasound image simultaneously.

15. The method of claim 11, further comprising highlighting regions of the first ultrasound image and/or the second ultrasound image based on the mask image.

16. The method of claim 1, further comprising receiving the first ultrasound image from an ultrasound device.

17. The method of claim 16, wherein receiving the first ultrasound image from the ultrasound device comprises receiving the first ultrasound image in real-time.

18. The method of claim 1, further comprising receiving the first ultrasound image from a memory.

19. The method of claim 1, wherein generating the second ultrasound image is performed in response to receiving a user selection.

20. The method of claim 1, wherein displaying the second ultrasound image is performed in response to receiving a first user selection.

21. The method of claim 20, wherein displaying the first ultrasound image is performed in response to receiving a second user selection following the first user selection.
